(19) United States
(12) Patent Application Publication
(10) Pub. No.: US 2013/0158640 A1
(43) Pub. Date: Jun. 20, 2013

(54) LEAD ANCHORING SYSTEM WITH LIMITED MOVEMENT OF ANCHORING DEVICE ALONG LEAD

(71) Applicants: Brian D. Soltis, St. Paul, MN (US); Kyle P. True, Coon Rapids, MN (US)

(72) Inventors: Brian D. Soltis, St. Paul, MN (US); Kyle P. True, Coon Rapids, MN (US)

(21) Appl. No.: 13/716,704

(22) Filed: Dec. 17, 2012

Related U.S. Application Data
(60) Provisional application No. 61/577,301, filed on Dec. 19, 2011.

(51) Int. Cl. A61N 1/05

(52) U.S. Cl. CPC A61N 1/057
USPC 607/116

(57) ABSTRACT

Described is a medical system for implantation within a patient's body. The medical system comprises: an implantable medical device; a lead having a proximal end, a distal end and an intermediate portion located between the proximal and distal ends, wherein the proximal end has a connector connecting the lead to the implantable medical device, and the intermediate portion includes a portion having a reduced outer diameter compared to an outer diameter of a remainder of the intermediate portion; and an anchoring device for anchoring the lead within the patient's body, the anchoring device comprising a sleeve having an inner lumen extending therethrough that accommodates the reduced outer diameter portion of the lead, wherein axial movement of the anchoring device along the lead is limited to within the reduced outer diameter portion of the lead.

### CROSS-REFERENCE TO RELATED APPLICATION

This application claims priority to Provisional Application No. 61/577,301, filed Dec. 19, 2011, which is herein incorporated by reference in its entirety.

### TECHNICAL FIELD

The present invention relates generally to implantable medical systems for anchoring medical leads to tissue of a patient. More specifically, the present invention relates to a lead anchoring system with limited movement of an anchoring device along a lead.

### BACKGROUND

Medical leads are anchored to a patient's tissue in a variety of applications using anchoring devices, including those commonly referred to as “suture sleeves.” For example, in many cardiac applications, an electrical lead connected to a cardiac rhythm management (CRM) device, such as a pacemaker, is secured to patient tissue at a vein entry site to help prevent both acute and chronic lead migration and dislodgement. In addition, in nerve stimulation applications, an electrical lead with electrodes placed near the nerve to be stimulated is connected to a pulse generator that is implanted elsewhere in the body. Such leads may be secured in place to prevent migration and dislodgement by securing a suture sleeve about the insulation of the lead and suturing the suture sleeve to the patient's tissue.

### SUMMARY

Some aspects of the invention relate to lead anchoring systems with limited movement of an anchoring device along a lead. The anchoring system includes an anchoring device that is securable to an implantable lead, such as those, for example, associated with cardiac management devices.

In Example 1, a medical system for implantation within a patient's body. The medical system includes an implantable medical device, a lead, and an anchoring device. The lead includes a proximal end, a distal end and an intermediate portion located between the proximal and distal ends. The proximal end has a connector connecting the lead to the implantable medical device. The intermediate portion includes a portion having a reduced outer diameter compared to an outer diameter of a remainder of the intermediate portion. The anchoring device for anchoring the lead within the patient's body, includes a sleeve having an inner lumen extending therethrough that accommodates the reduced outer diameter portion of the lead, wherein axial movement of the anchoring device along the lead is limited to within the reduced outer diameter portion of the lead.

In Example 2, the medical system according to Example 1, wherein the inner lumen of the anchoring device has a diameter that is less than the outer diameter of the remainder of the intermediate portion of the lead.

In Example 3, the medical system according to Examples 1 or 2, wherein the anchoring device includes at least one protrusion that extends inward into the inner lumen such that axial movement of the protrusion along the lead is limited to within the reduced outer diameter portion of the lead.

In Example 4, the medical system according to Examples 1-3, wherein the at least one protrusion inhibits rotational movement of the anchoring device around the lead.

In Example 5, the medical system according to Examples 1-4, wherein the at least one protrusion compresses the reduced outer diameter portion of the lead.

In Example 6, the medical system according to Examples 1-5, wherein the at least one protrusion extends around the circumference of the inner lumen of the anchoring device at a location along the reduced outer diameter portion of the lead.

In Example 7, an implantable medical system that includes a lead, and an anchoring device. The lead includes a proximal end, a distal end and an intermediate portion located between the proximal and distal ends. The intermediate portion includes a portion having a reduced outer diameter compared to an outer diameter of a remainder of the intermediate portion. The anchoring device, for anchoring the lead within the patient's body, includes a sleeve having an inner lumen extending therethrough that accommodates the reduced outer diameter portion of the lead, wherein axial movement of the anchoring device along the lead is limited to within the reduced outer diameter portion of the lead.

In Example, 8, the system according to Example 7, wherein the inner lumen of the anchoring device has a diameter that is less than the outer diameter of the remainder of the intermediate portion of the lead.

In Example 9, the system according to Example 7 or 8, wherein the anchoring device includes at least one protrusion that extends inward into the inner lumen such that axial movement of the protrusion along the lead is limited to within the reduced outer diameter portion of the lead.

In Example 10, the system according to Examples 7-9, wherein the at least one protrusion inhibits rotational movement of the anchoring device around the lead.

In Example 11, the system according to Examples 7-10, wherein the at least one protrusion compresses the reduced outer diameter portion of the lead.

In Example 12, the system according to Examples 7-11, wherein the at least one protrusion extends around the circumference of the inner lumen of the anchoring device at a location along the reduced outer diameter portion of the lead.

In Example 13, a method of anchoring a lead. The method includes the step of: providing a lead having a proximal end, a distal end and an intermediate portion located between the proximal and distal ends, wherein the intermediate portion includes a portion having a reduced outer diameter compared to an outer diameter of a remainder of the intermediate portion. The method also includes the step of: positioning an anchoring device for anchoring the lead in a patient's body about the reduced outer diameter portion of the lead, the anchoring device comprising a sleeve having an inner lumen extending therethrough that accommodates the reduced outer diameter portion of the lead, wherein axial movement of the anchoring device along the lead is limited to within the reduced outer diameter portion of the lead.

In Example 14, the method according to Example 13, further comprising the step of securing the anchoring device to tissue in a patient's body.

In Example 15, the method according to Example 13 or 14, further comprising the step of tying sutures around the anchoring device to secure the anchoring device to the lead.

In Example 16, the method according to Examples 13-15, wherein the inner lumen of the anchoring device has a diameter that is less than the outer diameter of the remainder of the intermediate portion of the lead.

In Example 17, the method according to Examples 13-16, wherein the anchoring device includes at least one protrusion that extends inward into the inner lumen such that axial movement of the protrusion along the lead is limited to within the reduced outer diameter portion of the lead.

In Example 18, the method according to Examples 13-17, wherein the at least one protrusion inhibits rotational movement of the anchoring device around the lead.

In Example 19, the method according to Examples 13-18, wherein the at least one protrusion compresses the reduced outer diameter portion of the lead.

In Example 20, the method according to Examples 13-19, wherein the at least one protrusion extends around the circumference of the inner lumen of the anchoring device at a location along the reduced outer diameter portion of the lead.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic view of a medical system implanted in a patient's body (designated generally by a circle in FIG. 1), according to various embodiments of the invention.

FIG. 2 is a front view of an anchoring system of the medical system of FIG. 1, according to some embodiments.

FIG. 3 is a cross-sectional view of the anchoring system of FIG. 2 taken along line 3-3 of FIG. 2.

FIGS. 4A and 4B are two exemplary cross-sectional views of the anchoring system of FIG. 2 taken along line 4-4 of FIG. 2.

FIG. 5 is a front view of another anchoring system of the medical system of FIG. 1, according to some embodiments.

FIG. 6 is a cross-sectional view of the anchoring system of FIG. 5 taken along line 6-6 of FIG. 5.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

Various embodiments described herein relate to lead anchoring devices with limited movement of the anchoring device along a lead. The anchoring device can be securable to an implantable lead, for example, associated with cardiac management devices. Other uses for the anchoring device are contemplated, however, such as in vagus nerve stimulation systems. Other suitable uses for the anchoring device described, however, are also contemplated.

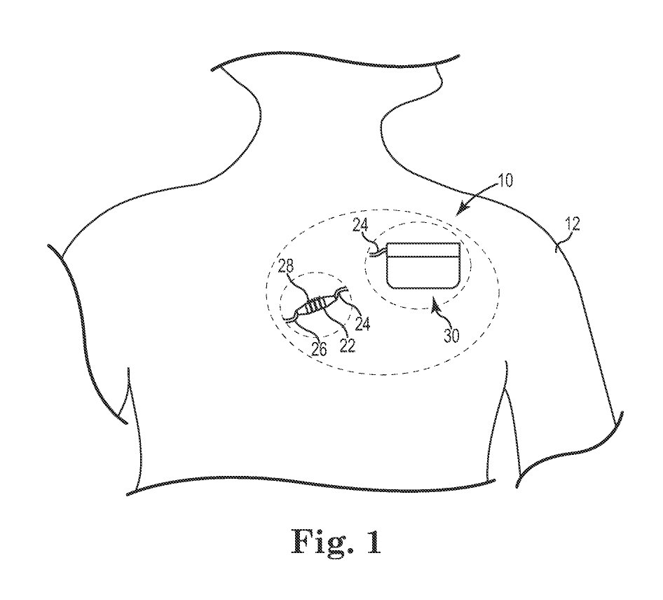

FIG. 1 is a simplified view of a medical system 10 implanted in a patient's body 12 according to various embodiments of the present disclosure. The system 10 includes an anchoring device 22 (e.g., suture sleeve), a lead 24, and an implantable medical device (IMD) 30 connected to the lead 24.

The IMD 30 can be, for example, a pulse generator, a pacemaker, an implantable cardioverter/defibrillator (ICD), a cardiac resynchronization therapy (CRT) device or another therapeutic device (e.g., drug pump) implanted in the patient's body 12. The IMD 30 can be implanted subcutaneously or submuscularly within an implantation location, or pocket, in the patient's chest or abdomen, for example. The IMD 30 may be an implantable medical device known in the art or later developed, for delivering an electrical therapeutic stimulus to a patient.

In some embodiments, the lead 24 can be an electrical lead of a type suitable for use with CRM devices, for example. The lead 24 can include one or more inner conductors (not shown) or other internal features and an outer, insulating sheath 26 extending over the internal features of the lead 24. In some embodiments, the lead 24 can include electrodes (not shown) or other features for stimulating or sensing functionality.

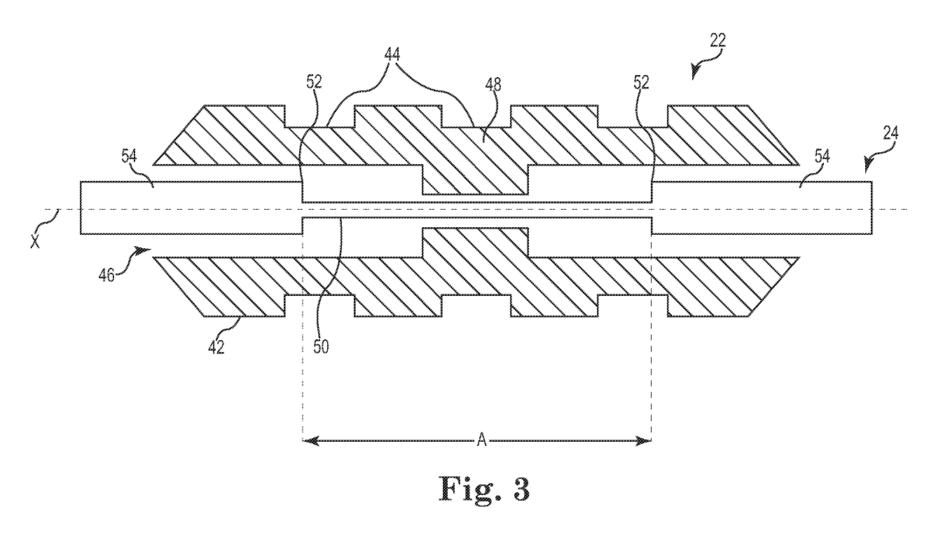

The lead 24 also includes a portion having a reduced outer diameter 50 compared to the remainder of the lead 24. The portion having a reduced outer diameter 50 of the lead 24 has a length corresponding to limited distance A, which is the distance that the lead 24 is able to move through or within anchoring device 22. Ends 52 of the non-reduced lead portions 54 are indicated as 52 in FIG. 3. These ends 52 act as physical hard stops to movement of lead 24 though anchoring device 22 as one of the two ends 52 contacts protrusion 48 (discussed herein in more detail). The reduction in the cross-sectional area or outer diameter of the lead 24 in area 50 is reduced enough to allow the reduced portion 50 of the lead 24 to move through anchoring device 22 in the area of protrusion 48. However, the diameter or cross-sectional area of lead 24 at reduced portion 50 must also be sufficiently large to allow conductors or other lead components (not shown) to run through or extend through lead 24 and function properly, without damage being done to such lead components or conductors during implantation and/or suturing.

As shown in FIG. 1, and in general terms, the anchoring device 22 is positioned over the insulating sheath 26 of the lead 24 and can serve to stabilize the lead 24 at or near an entry site (not shown), for example, to help prevent both acute and chronic lead migration and dislodgement. As discussed further herein, the anchoring device 22 is configured such that movement of the anchoring device 22 along the lead 24 is limited. By limiting the movement of the anchoring device 22 with respect to the lead 24, the anchoring device 22 can be prevented from sliding down the lead 24 into a vein or sliding off the lead 24 completely. The limited movement can also keep the anchoring device 22 accessible for the physician, for example, during implantation of the medical system 10.

In some embodiments, once the anchoring device 22 is at a desired location, the anchoring device 22 may be compressed onto the lead 24 using sutures 28 secured about the anchoring device 22. According to some embodiments, the sutures 28 can be manually secured about the anchoring device 22 by a physician using some tension or tying force.

The anchoring device 22 may include other suturing structures for securing the anchoring device 22 to tissue. For example, radially projecting fins or tabs may be provided as suturing structures. In addition, other suturing structures can also be used for securing anchoring device 22 to tissue.

Although the sutures 28 can be used to compress the anchoring device 22 onto the lead 24 for lead retention, in other embodiments, the anchoring device 22 may be sufficiently “self-restraining” on the lead 24 such that the compressive forces from the sutures 28 are not necessary to secure the anchoring device 22 to the lead 24 for long term implantation (though the sutures 28 may still be present to secure the anchoring device 22 to adjacent tissue of the patient's body 12). Additionally, although sutures, in general, and manual methods of tying of sutures 28, in particular, are referenced herein, other fastening means and methods, such as spring clips or automatic suture tying devices are contemplated.

The anchoring device 22 can be adapted for improved lead retention that has a predetermined magnitude that is substantially consistent and independent of user technique. The anchoring device 22 also can act to minimize deformation or other damage to the insulating sheath 26 or other internal features of the lead 24 (e.g., conductive coils). For example, in some instances, the conductors and/or insulative sheath 26 can be damaged by concentrated radial forces at the interface between the lead 24 and the anchoring device 22 proximate the sutures 28 if insufficiently protected.

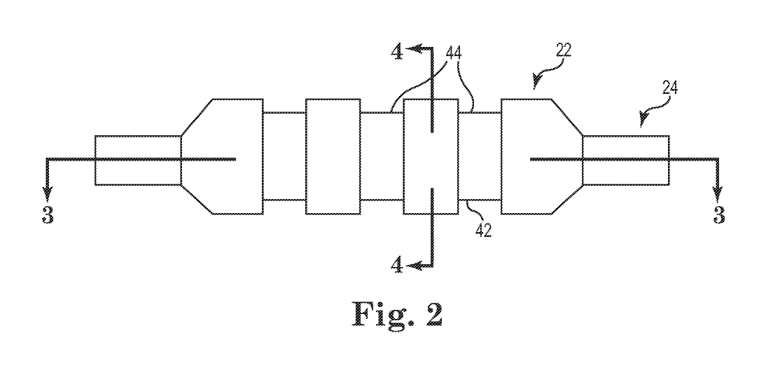

FIG. 2 shows anchoring device 22 and a portion of lead 24 from a side view, according to some embodiments. As shown, the anchoring device 22 has an outer surface 42 and at least one suture groove 44 (three suture grooves are shown in FIG. 2) in the outer surface 42. Alternatively, the anchoring device 22 can have tabs, fins, wings or some other protrusion or feature that may serve as a means to fix the anchoring device 22 to tissue adjacent to the anchoring device 22 when placed in a patient. The anchoring device 22 also includes an inner lumen 46 (see FIG. 3) adapted to receive and maintain the lead 24.

FIG. 3 is a longitudinal cross-sectional view of the anchoring device 22 taken along line 3-3 of FIG. 2.

According to some embodiments, the anchoring device 22 is formed of an elastomeric material and is generally flexible, substantially compliant, and elastically compressible. For example, anchoring device 22 may be formed of silicones and polyurethanes or other biocompatible material having suitable properties. The anchoring device 22 may be formed via a variety of forming methods, including, for example, insert molding.

As shown in FIGS. 2 and 3, the outer surface 42 of anchoring device 22 has a plurality of circumferentially extending suture grooves 44 formed into the outer surface 42. The anchoring device 22 also can include features such as longitudinal slots (not shown) for facilitating compression of the anchoring device 22.

Before being attached to tissue, the anchoring device 22 can move along lead 24 through a limited distance, zone or region. In some embodiments, the anchoring device 22 can be provided with a limited distance, zone or a region of the lead along which the anchoring device 22 may move, so that the anchoring device 22 may be attached to tissue anywhere at or near the limited distance, zone or region. The limited distance, zone or region can allow the anchoring device 22 to be adjustable for different anatomies of patients and for different applications in the body. An exemplary limited distance is indicated by A in FIG. 3, although other lengths of distances are also possible. The anchoring device 22 is able to move along lead 24 the limited distance A as a result of the configuration of both the anchoring device 22 and the lead 24. The distance A provides some adjustability to the device 22 to account for differences in anatomies of patients or regions of the body in which the device 22 is implanted. The limited distance A also prevents the anchoring device 22 from moving down the lead 24 to an undesired position or location, or even from falling off the end of the lead 24.

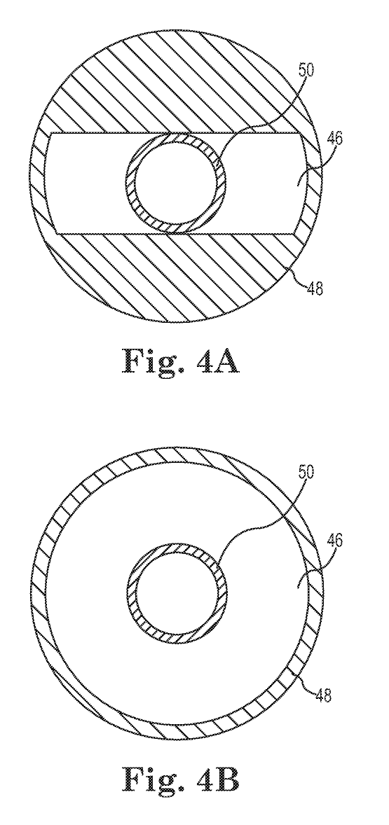

Anchoring device 22 includes at least one protrusion 48 that extends into an inner lumen 46 of anchoring device 22. The protrusion 48 can result in reducing the diameter or cross-sectional area of the inner lumen 46 in the area of the protrusion 48. The protrusion 48 may have a suitable shape or configuration, with two examples being shown in FIGS. 4A and 4B, and discussed in more detail herein. The protrusion 48, or reduction of the cross-sectional area or diameter compared to the remainder of the lumen 46, provides a stop for movement of the lead 24 through or within the lumen 46. The protrusion 48, together with the portion 50 of the lead 24 having a reduced outer diameter, limits the distance that the anchoring device 22 is able to move along the lead 24 (which is limited to limited distance A in the figure).

Two possible shapes for protrusion 48 of anchoring device 22 are shown in FIGS. 4A and 4B, although other shapes are also contemplated. Both exemplary shapes show the protrusion 48 extending around the circumference of the inner lumen of the anchoring device. However, it is not necessary that the protrusion 48 extend around the entire circumference of the inner lumen.

FIG. 4A shows a cross-section of anchoring device 22 viewed from 4-4 in FIG. 2. In FIG. 4A, protrusion 48 is shaped such that inner lumen 46 has an opening with a rectangular shape. The protrusion 48 limits longitudinal movement of lead 24 by acting as a physical hard stop to ends 52 of lead 24 (as shown in FIG. 3). Also, protrusion 48 may limit rotational movement of lead 24. As shown in FIG. 4A, lead 24 (at reduced portion 50, as shown in FIG. 3) can be slightly compressed by protrusion 46, which may prevent or inhibit rotational movement of the lead 24 within anchoring device 22.

FIG. 4B shows another exemplary cross-section of anchoring device 22 and another exemplary shape of protrusion 48. The shape of protrusion 48 is circular. As shown, protrusion 48 may not contact lead 24 (in reduced portion 50, as shown in FIG. 3), but may act as a physical hard stop to ends 52 of lead 24 (as shown in FIG. 3). The protrusion 48 shown in FIG. 4B does not, however, prevent or inhibit rotational movement of lead 24 within anchoring device 222.

Besides the protrusion 48 in inner lumen 46 of anchoring device 22, inner lumen 46 may be smooth-bored in some embodiments, as shown in FIGS. 4A and 4B. Alternatively, the inner lumen 46 may include additional roughening or other friction enhancing features.

Once the anchoring device 22 is in a desired implantation location, the sutures 28 (FIG. 1) are aligned to the suture grooves 44 and tightened about the anchoring device 22 to secure the anchoring device 22 to surrounding tissue of the patient's body 12 (FIG. 1) and also the lead 24. Alternatively, the anchoring device 22 may be secured to adjacent tissue or the lead using other means of fixation, such as those described herein.

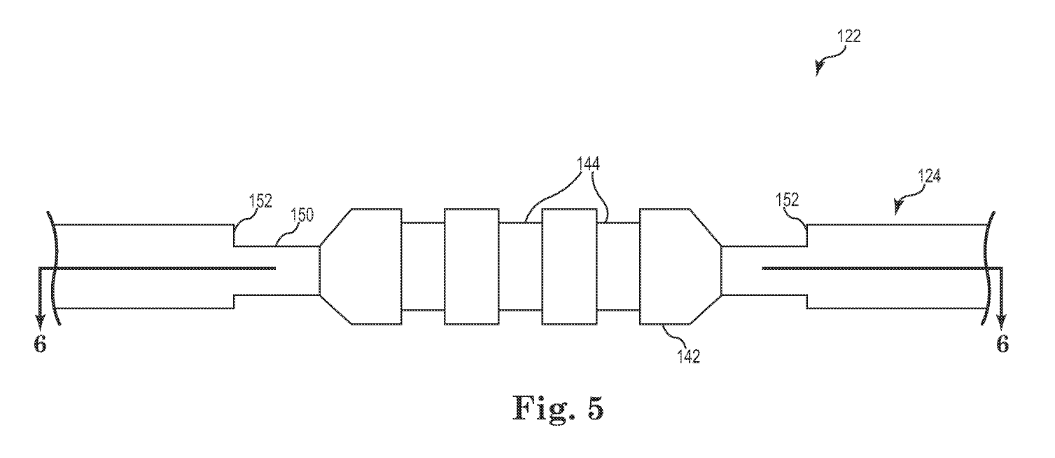

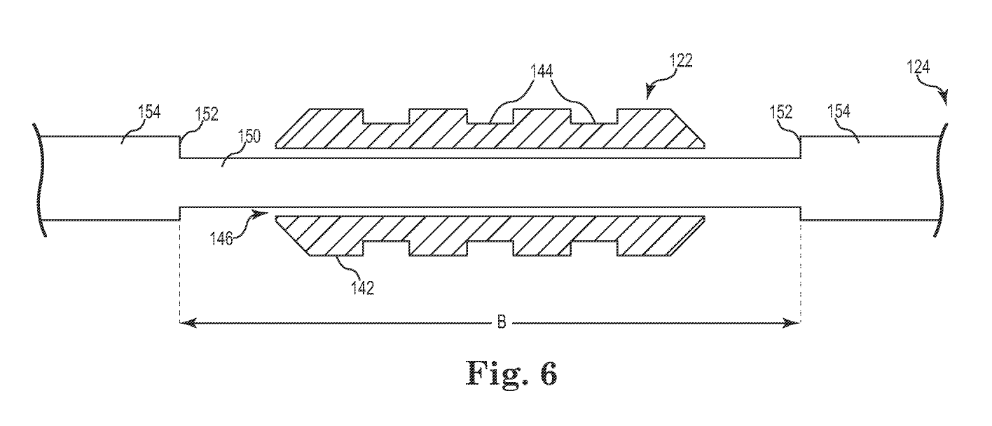

FIGS. 5 and 6 illustrate another alternative anchoring device 122 suitable for use in medical system 10. FIG. 5 shows anchoring device 122 and a portion of a lead 124 from a side view, according to some embodiments. FIG. 6 is a cross-sectional view of anchoring device 122 viewed from line 6-6 in FIG. 5. Anchoring device 122 is optionally used in a substantially similar manner, according to substantially similar methods, to the anchoring device 22 to secure lead 124 within a patient's body.

As shown, lead 124 (similar to lead 24 in FIG. 3) includes a portion having a reduced outer diameter 150 compared to the remainder of the lead 124. The reduced area 150 of the lead 124 is indicated by length B (see FIG. 6). Length B is the limited distance, zone or region of adjustability of the anchoring device 122. Anchoring device 122 rides along lead 124 in the reduced outer diameter portion 150 of lead 124. An inner lumen 146 of anchoring device 122 (as shown in FIG. 6) is adapted to receive lead 124. Anchoring device 122 also has an outer surface 142 and at least one suture groove 144 (three suture grooves are shown) in the outer surface 142. Alternative means may be used to the suture grooves 144, however, in order to secure the anchoring device 122 to adjacent tissue.

Anchoring device 122 is only able to move along lead 124 within distance B. The limited distance, zone or region B, prevents the anchoring device 122 from falling off the lead 124 during implantation, for example. Ends 152 of non-reduced lead portions 154 can act as physical hard stops to movement of anchoring device 122 along lead 124.

Although the anchoring device (22 and 122) has been described in association with lead (24 and 124), it should be understood that in some embodiments the device (24 and 124) is used to secure a tube or any other implantable elongated member. For example, the lead (24 and 124) is optionally substituted with a tube, cord, conduit, duct, wire, cable, fiber optics, or any other substantially similar implantable item rather than a medical device lead.

A method of anchoring a lead is also disclosed. First, a lead is provided having a proximal end, a distal end and an intermediate portion located between the proximal and distal ends, wherein the intermediate portion includes a portion having a reduced outer diameter compared to an outer diameter of a remainder of the intermediate portion. The anchoring device is then positioned in a patient's body about the reduced outer diameter portion of the lead, the anchoring device comprising a sleeve having an inner lumen extending therethrough that accommodates the reduced outer diameter portion of the lead. Axial movement of the anchoring device along the lead is limited to the reduced outer diameter portion of the intermediate portion of the lead. The anchoring device may include a protrusion that fits within the reduced outer diameter portion of the lead. Alternatively, the anchoring device may fit within the reduced outer diameter portion of the lead. Once in a desired position, the anchoring device may be secured to tissue in a patient's body. The method is exemplary, however, and other methods are also contemplated.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A medical system for implantation within a patient's body, the medical system comprising:
an implantable medical device;
a lead having a proximal end, a distal end and an intermediate portion located between the proximal and distal ends, wherein the proximal end has a connector connecting the lead to the implantable medical device, and the intermediate portion includes a portion having a reduced outer diameter compared to an outer diameter of a remainder of the intermediate portion; and
an anchoring device for anchoring the lead within the patient's body, the anchoring device comprising a sleeve having an inner lumen extending therethrough that accommodates the reduced outer diameter portion of the lead, wherein axial movement of the anchoring device along the lead is limited to within the reduced outer diameter portion of the lead.

2. The medical system of claim 1, wherein the inner lumen of the anchoring device has a diameter that is less than the outer diameter of the remainder of the intermediate portion of the lead.

3. The medical system of claim 1, wherein the anchoring device includes at least one protrusion that extends inward into the inner lumen such that axial movement of the protrusion along the lead is limited to within the reduced outer diameter portion of the lead.

4. The medical system of claim 3, wherein the at least one protrusion inhibits rotational movement of the anchoring device around the lead.

5. The medical system of claim 4, wherein the at least one protrusion compresses the reduced outer diameter portion of the lead.

6. The medical system of claim 3, wherein the at least one protrusion extends around the circumference of the inner lumen of the anchoring device at a location along the reduced outer diameter portion of the lead.

7. An implantable medical system comprising:
a lead having a proximal end, a distal end and an intermediate portion located between the proximal and distal ends, wherein the intermediate portion includes a portion having a reduced outer diameter compared to an outer diameter of a remainder of the intermediate portion; and
an anchoring device for anchoring the lead within the patient's body, the anchoring device comprising a sleeve having an inner lumen extending therethrough that accommodates the reduced outer diameter portion of the lead, wherein axial movement of the anchoring device along the lead is limited to within the reduced outer diameter portion of the lead.

8. The system of claim 7, wherein the inner lumen of the anchoring device has a diameter that is less than the outer diameter of the remainder of the intermediate portion of the lead.

9. The system of claim 7, wherein the anchoring device includes at least one protrusion that extends inward into the inner lumen such that axial movement of the protrusion along the lead is limited to within the reduced outer diameter portion of the lead.

10. The system of claim 9, wherein the at least one protrusion inhibits rotational movement of the anchoring device around the lead.

11. The system of claim 10, wherein the at least one protrusion compresses the reduced outer diameter portion of the lead.

12. The system of claim 9, wherein the at least one protrusion extends around the circumference of the inner lumen of the anchoring device at a location along the reduced outer diameter portion of the lead.

13. A method of anchoring a lead comprising the steps of:
providing a lead having a proximal end, a distal end and an intermediate portion located between the proximal and distal ends, wherein the intermediate portion includes a portion having a reduced outer diameter compared to an outer diameter of a remainder of the intermediate portion;
positioning an anchoring device for anchoring the lead in a patient's body about the reduced outer diameter portion of the lead, the anchoring device comprising a sleeve having an inner lumen extending therethrough that accommodates the reduced outer diameter portion of the lead, wherein axial movement of the anchoring device along the lead is limited to within the reduced outer diameter portion of the lead.

14. The method of claim 13, further comprising the step of securing the anchoring device to tissue in a patient's body.

15. The method of claim 13, further comprising the step of tying sutures around the anchoring device to secure the anchoring device to the lead.

16. The method of claim 13, wherein the inner lumen of the anchoring device has a diameter that is less than the outer diameter of the remainder of the intermediate portion of the lead.

17. The method of claim 13, wherein the anchoring device includes at least one protrusion that extends inward into the inner lumen such that axial movement of the protrusion along the lead is limited to within the reduced outer diameter portion of the lead.

18. The method of claim 17, wherein the at least one protrusion inhibits rotational movement of the anchoring device around the lead.

19. The method of claim 18, wherein the at least one protrusion compresses the reduced outer diameter portion of the lead.

20. The method of claim 17, wherein the at least one protrusion extends around the circumference of the inner lumen of the anchoring device at a location along the reduced outer diameter portion of the lead.
